(19) United States
(12) Patent Application Publication
(10) Pub. No.: US 2011/0015123 A1
(43) Pub. Date: Jan. 20, 2011

(54) Modulation of GLUT4 Gene Promoter activity BY AHNAK

(75) Inventors: Eddy Karnieli, Kiryat-Tivon (IL); Dafna Ben-Yosef, Naharya (IL)

Correspondence Address: BROWDY AND NEIMARK, P.L.L.C.;624 NINTH STREET, NW, SUITE 300, WASHINGTON, DC, 20001-5303, US

(73) Assignee: Technion Research and Developmet Foundation Ltd., Haifa (IL)

(21) Appl. No.: 12/423,440

(22) Filed: Apr. 14, 2009

Related U.S. Application Data
(60) Provisional application No. 61/044,735, filed on Apr. 14, 2008.

(51) Int. Cl. A61K 38/02; C07K 2/00; C07H 21/04; C12N 15/63; A61P 3/10; A61P 3/04; A61P 9/12; A61K 31/713; A61P 5/50; C12Q 1/02

(52) U.S. Cl. 514/4.8; 530/300; 536/24.5; 435/320.1; 514/6.7; 514/44 A; 435/29

(57) ABSTRACT

Agents capable of alleviating AHNAK phosphoprotein-mediated repression of GLUT4 gene expression are useful for prevention, treatment, and/or alleviation of insulin resistance associated with obesity, lipotoxicity, hypertension, metabolic syndrome and type 2 diabetes. Preferred agents are double-stranded siRNAs.

### FIELD OF THE INVENTION

The present invention relates to AHNAK-mediated GLUT4 gene promoter repression, to agents, such as antisense against the AHNAK gene, peptides or chemicals, capable of alleviating said repression, and to a method for detecting new modulators of such repression. Since GLUT4 mediates insulin responsive glucose transport, AHNAK is introduced as a novel molecular target for therapy in insulin resistance states like obesity and diabetes.
Abbreviations: ChIP, chromatin immunoprecipitation; DME—Dulbecco's Modified Eagle's (medium); FFA, free fatty acids; GLUT4-P, GLUT4 gene promoter; IRE, insulin response element; PRA—primary rat adipocytes; pC-DY, AHNAK C-terminus; pM-DY, AHNAK middle part; pN-DY, AHNAK N-terminus.

### BACKGROUND OF THE INVENTION

Glucose uptake in eukaryotic cells is mediated by glucose transport proteins. GLUT4, the insulin-regulated glucose transporter, mediates glucose uptake in response to insulin stimuli, by that playing the major rate limiting step in glucose metabolism. GLUT4 is found in adipose tissue and striated muscle. The expression and function of the GLUT4 isoform has been extensively studied, and found to be tightly regulated at both mRNA and protein levels (Armoni et al., 2007). Its expression is lower in hyperlipidemia and diabetes mellitus type 2 (DM2), hence causing insulin resistance state.

Obesity, lipotoxicity, Diabetes Mellitus type 2, insulin resistance, and metabolic syndrome are leading morbidities in today's western world, with a prevalence rapidly rising

Insulin resistance and hyperlipidemia play a central role in obesity, type 2 diabetes mellitus, and metabolic syndrome (Petersen and Shulman, 2006), and are associated with elevation of plasma free fatty acid (FAA) levels (Boden, 1997). While prolonged exposure to high FFA levels lead to insulin resistance in insulin responsive tissues such as skeletal muscle and cardiac muscle (Griffin et al., 1999; Yu et al, 2002) the mechanisms involved are yet unclear.

Apart from their metabolic role, FFAs modulate gene transcription by exerting a direct, membrane-independent influence on the molecular events that govern gene expression, mRNA stability and cellular differentiation (Armoni et al., 2007). The critical importance of tissue-specific regulation of GLUT4 for maintaining normal glucose homeostasis is amplified in altered metabolic states. With the development of insulin resistance, GLUT4 expression is down-regulated selectively in adipose tissue but not in skeletal muscle (Shepherd and Kahn. 1999). Type 2 diabetes mellitus, obesity, aging and the metabolic syndrome are associated with marked reduction of GLUT4 gene expression in adipose cells, which in turn lead to insulin resistance Impaired insulin stimulation of glucose transport in adipose tissue and muscle is one of the earliest defects detected in insulin-resistant states (Shepherd and Kahn 1999).

Obesity is associated with insulin resistance (Boden, 1997) and with inflammation, which is characterized by elevation of pro-inflammatory cytokines in blood and tissues (Tataranni and Ortega 2005). Plasma FFA levels are elevated in obesity initially because the enlarged adipose tissue mass releases more FFA.

Elevated concentrations of plasma FFA in general and arachidonic acid in particular, inhibit insulin-stimulated glucose uptake into muscle. Several mechanisms have been proposed for FFAs-induced insulin resistance, among them: reduced phosphorylation of insulin receptor substrate (IRS)-1 by activating protein kinase C_{θ} (Yuan et al., 2001); attenuation of GLUT4 translocation through activation of the IκB kinase pathway (Yuan et al., 2001); reduction of cellular GLUT4 gene expression at transcriptional and post transcriptional level (Armoni et al, 2005); and diminution of intrinsic activity of GLUT4 (Alkhateeb et al., 2007).

Genetic modulation of GLUT4 at cellular level has been shown to affect whole body glucose homeostasis. Thus, mice over-expressing GLUT4 either systemically or specifically in either skeletal muscle or adipose tissue, displayed enhanced insulin responsiveness and peripheral glucose utilization, even in experimental diabetes (Charron et al., 1999). Further, in adipose-specific GLUT4 null mice reduction in cellular GLUT4 levels contributed to insulin resistance in obesity and diabetes, probably via altering the release of novel adipocyte-secreted molecules such as the retinol-binding protein 4 (Graham et al., 2006).

In C2Cl2 myoblasts, chronic exposure to palmitic acid resulted in increased mRNA and protein levels of the proinflammatory cytokine TNF-α, in correlation with decreased GLUT4 expression and PKC- and NFκB-dependent functions (Jove et al. 2006). FFA also inhibited insulin-induced eNOS (Endothelial Nitric Oxide Synthase) activation (Wang et al., 2006), while IRS-mediated serine phosphorylation by phosphoinositide 3-kinase (PI3K) is impaired following lipid infusion (Yu et al., 2002). Thus, it appears that FFA may exert their detrimental effects via several mechanisms/mediators.

Recently, it has been shown that GLUT4 protein levels are 30% lower in cardiac muscle of patients with hyperlipidemia and/or type 2 diabetes mellitus compared with euglycemic control subjects, and that high FFA levels reduce transcription from the GLUT4 gene promoter (Armoni et al. 2005). Several potential cis-elements on the GLUT4 promoter were identified, which may mediate its repression of GLUT4 gene expression by FFA. However, the endogenous product(s) mediating this effect remained illusive.

AHNAK/desmoyokin is a giant phosphoprotein playing a role in glucose homeostasis by mediating GLUT4 gene repression by arachidonic acid. Its levels were shown to be increased in adipose and muscle tissues in insulin resistance states like obesity. There is evidence that AHNAK is involved in: arachidonic acid metabolism via PLCγ (Lee et al., 2004) activation of RAF-MEK-ERK signaling pathway (Lee et al., 2008), calcium signaling by interaction with calcium channels in the plasma membrane (Haase et al., 1999); skeletal muscle regeneration (Huang et al., 2007); and cytoskeleton interaction via actin (Hohaus et al., 2002).

Data indicating AHNAK association with membrane calcium channels and actin-based cytoskeletal functions (Hohaus et al., 2002) imply that it may affect signaling pathways mediated via calcium and the extracellular matrix (ECM) in cellulo.

Lately, it has been shown that AHNAK localization can either be nuclear, cytoplasmic or membrane-bound. In the nucleus, AHNAK can weakly bind to the DNA (Stiff et al., 2004). AHNAK translocation into the nucleus is mediated by protein kinase B (PKB) (also known as “Akt”, Akt1 or PKB/Akt) phosphorylation at AHNAK's C terminus-located nuclear export signal (NES). Furthermore, Sussman et al (2001) reported that nuclear exclusion of AHNAK is mediated via an export signal that requires phosphorylation of Ser-5335 by PKB/Akt. Thus, AHNAK is considered a PKB/Akt substrate. Plasma membrane localization of AHNAK has been observed in rodent and human cardiomyocytes as well as in smooth and skeletal muscles (Haase et al., 1999; Hohaus et al,. 2002; Gentil et al. 2003; Haase et al., 2004).

The use of cDNA subtraction screen for genes differentially expressed in epididymal fat pads harvested one week after the start of a 60% fat diet, showed that AHNAK gene expression is 6 times higher in adipose tissue at the early stage of high fat diet-induced obesity in rats (Li et al., 2002) than in normal rats, but its role in obesity/diabetes has not yet been studied.

Expression of genes in eukaryotic cells may be specifically repressed by interfering with the mRNA transcripts. For example, a long dsRNA (more than 30 nucleotides) that binds to the corresponding sequence of a particular single-stranded mRNA (ss mRNA) prevents translation of the mRNA by destabilizing that message, preventing binding to ribosomes or preventing translocation to the cytosol. When a shorter RNA binds to the target mRNA, interference is achieved by a different mechanism; double-stranded siRNAs (short inhibitory RNAs) are produced from dsRNA through the activity of an RNase III family endonuclease denoted Dicer. They are generally 21 to 23 nucleotide long and have overhanging ends consisting of two nucleotides. siRNAs interact with a large multi-component enzyme termed RNA-induced silencing complex (RISC), to bind a fully complementary mRNA sequence, which results in endonucleolytic cleavage of the mRNA. Accordingly, any selected gene may be repressed by introducing a dsRNA which corresponds to all or a substantial part of the mRNA for that gene.

Diverse eukaryotic species, including humans, possess non-coding regulatory endogenous hairpin RNAs, also known as microRNAs (miRNA), that can interact with RISC and silence genes. However, unlike siRNAs, miRNAs do not induce degradation of mRNA, but rather partially bind to its 3′ UTR and block translation. Thus, RISC can interfere with protein synthesis and this is the dominant mechanisms used by miRNAs in mammals. In addition, short RNAs can shut down gene expression by inducing specific methylation of promoters in several species including humans miRNA may be cloned into expression vectors under the control of appropriate promoters and used to silence specific genes similarly to siRNAs, or the expression of endogenous miRNAs may be induced by activation of their promoters.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

In one aspect, the present invention relates to agents and methods for prevention, treatment, alleviation or a combination thereof of insulin resistance associated with obesity, lipotoxicity, hypertension, metabolic syndrome and type 2 diabetes, comprising administering to an individual in need a therapeutically effective amount of an agent capable of alleviating AHNAK phosphoprotein-mediated repression of GLUT4 gene expression.

In one embodiment, the method comprises the prevention of insulin resistance associated with obesity. In another embodiment, the method comprises the treatment of insulin resistance associated with obesity. In a further embodiment, the method comprises the alleviation of insulin resistance associated with obesity.

In another embodiment, the method comprises the prevention of insulin resistance associated with lipotoxicity. In a further embodiment, the method comprises the treatment of insulin resistance associated with lipotoxicity. In yet a further embodiment, the method comprises the alleviation of insulin resistance associated with lipotoxicity.

In another embodiment, the method comprises the prevention of insulin resistance associated with hypertension. In a further embodiment, the method comprises the treatment of insulin resistance associated with hypertension. In yet a further embodiment, the method comprises the alleviation of insulin resistance associated with hypertension.

In another embodiment, the method comprises the prevention of insulin resistance associated with metabolic syndrome. In a further embodiment, the method comprises the treatment of insulin resistance associated with metabolic syndrome. In yet a further embodiment, the method comprises the alleviation of insulin resistance associated with metabolic syndrome.

In another embodiment, the method comprises the prevention of insulin resistance associated with type 2 diabetes. In a further embodiment, the method comprises the treatment of insulin resistance associated with type 2 diabetes. In yet a further embodiment, the method comprises the alleviation of insulin resistance associated with type 2 diabetes.

In a further aspect, the present invention relates to a method for detecting a modulator of AHNAK phosphoprotein-mediated repression of GLUT4 gene expression, as described below.

### BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A-1B show that arachidonic acid represses GLUT4 promoter activity in rat embryonal heart-derived H9C2 cells and primary rat adipocytes (PRA), respectively. Fully-differentiated rat H9C2 myotubes (1A) and PRA (1B) were transfected with GLUT4-P-Luc promoter reporter (2 μg DNA), and then incubated with ambient concentrations of arachidonic acid as indicated, for 24 hours. GLUT4 promoter activity was assessed based on the luciferase activity normalized to β-galactosidase in each sample, and expressed as a percentage of basal promoter activity. Each point represents an average of 3 experiments performed in triplicates±SEM.

FIGS. 2A-C show that AHNAK and arachidonic acid synergistically repress GLUT4 promoter activity in PRA. PRA were transfected with GLUT4-P-Luc promoter reporter (2 μg DNA) along with expression vectors (0-5 μg DNA) coding for AHNAK C-terminus pC-DY (2A), middle part pM-DY (2B) and N-terminus pN-DY (2C). Cells were then incubated with bovine serum albumin (BSA) or arachidonic acid (100 μM) for 24 hours. GLUT4 promoter activity was assessed based on the luciferase activity normalized to β-galactosidase in each sample, and expressed as a percentage of basal promoter activity. Each point represents an average of 3 experiments performed in triplicates±SEM.

FIGS. 3A-B depict progressive 5′-Deletion Analysis of GLUT4-P. The full-length GLUT4 promoter reporter and a series of progressive 5′-deleted mutants (3A, left panel) were generated as detailed in “Experimental Procedures”. The deletion points used to generate each construct are indicated and numbered. PRA were transiently co-transfected with the various promoter-reporters as indicated, along with either pC-DY, pM-DY or pN-DY (3B, right panel) expression vectors (2.5 μg DNA). GLUT4 promoter activity was assessed based on the luciferase activity normalized to β-galactosidase activity in each sample, as described in “Experimental Procedures”. For clarity, results are expressed as a percentage of each basal promoter activity in each construct. The data are expressed as mean±SEM of 4 experiments, with each sample analyzed in triplicate.

FIG. 4 shows that the AHNAK middle part binds the −222/−197 bp region on GLUT4-P in cellulo. The results represent chromatin immunoprecipitation (ChIP) analysis preformed in fully-differentiated rat H9C2 myotubes transfected with either pC-DY, pM-DY, or pN-DY expression vectors (5 μg DNA). Forty-eight hours post-transfection, cells were subjected to two-step cross-linking, then lysed and sonicated as described under “Experimental Procedures.” An aliquot of the whole cell lysate (WCL) was removed for purification of total DNA, and immunoprecipitations were conducted using either anti-cMyc (myc) antibody or anti-IgG (IgG) antibodies. DNA was extracted from the immunoprecipitates, and PCR (26 cycles) was conducted on total DNA and immunoprecipitated DNA with primers corresponding to promoter region −222/−197 of the rat GLUT4 promoter. PCR products were analyzed on 2% agarose gel and visualized by ethidium bromide staining in the presence of DNA molecular mass markers.

FIGS. 5A-C show the effects of AHNAK silencing on GLUT4 gene expression. PRA were transfected by electroporation with non targeting (NT) or AHNAK siRNA. After transfection, cells were either incubated with BSA or arachidonic acid (100 μM) for 24 hrs. RNA and protein lysate were extracted and AHNAK mRNA (5A, left panel) and GLUT4 protein level (5B, upper right panel) were assessed by quantitative real time (QRT)-PCR and Western blotting analyses, respectively. Assays for β-actin internal control were run in parallel for each sample (5C, lower right panel).

FIGS. 6A-6B show that there is cross talk between AHNAK and insulin signaling components. PRA were transfected with either the synthetic promoter reporter 3×IRS-LUC (6A) or GLUT4-P-Luc (6B), along with pC-DY, pM-DY, or pN-DY expression vectors (2.5 μg DNA). Cells were then incubated in presence of either DMSO for control, insulin alone or combined with wortmannin or AKT inhibitor for 24 hrs. GLUT4 promoter activity was measured based on the luciferase activity normalized to β-galactosidase in each sample. For clarity, results are expressed as a percentage of the basal promoter activity. Each point in the figure represents average of results from 3 experiments performed in triplicates±SEM.

FIG. 7 shows AHNAK cellular distribution in HEK cells. HEK cells were chosen mostly due to easy staining procedure, and were incubated for 24 hrs in a serum free medium containing either insulin (100 nM) alone (middle panels) or combined with LY (20 μM, lower panels). Insulin free medium was used for control (upper panels). After 24 hrs, endogenous AHNAK was stained with DAPI by immunoflorescence.

FIG. 8 depicts a suggested model for AHNAK and arachidonic acid regulation of GLUT4 gene expression. Based on the data we obtained from 5′-del and ChIP analyses on one hand, and siRNA analysis on the other, we suggest a model for AHNAK and arachidonic acid regulation of GLUT4 gene expression, that consequently leads to impaired insulin sensitivity in PRA. See text for detailed description.

### DETAILED DESCRIPTION OF THE INVENTION

In the quest to define the mediator of repression of GLUT4 gene expression by free fatty acids (FFA0 we used mass spectrometry analysis and discovered that AHNAK/desmoyokin, a giant phosphoprotein, is associated with the GLUT4-P region −222/−197 bp (see Example 1).

We then examined the effects of AHNAK on basal GLUT4 gene expression, as well as its effect during chronic exposure to high FFA levels that simulate hyperlipidemia, obesity and diabetic states. We identified AHNAK as a potential mediator of arachidonic acid-induced GLUT4 repression, operating via direct binding to region −222/−197 bp of the GLUT4 promoter.

In accordance with the present invention, an agent is provided that alleviates the AHNAK-mediated repression of GLUT4 gene expression. The agent alleviates repression of GLUT4 gene by direct and/or indirect influence on AHNAK mediator. As such, the agent represents a novel approach for prevention and treatment of insulin resistance states associated with obesity, lipotoxicity, metabolic syndrome and type 2 diabetes mellitus. This novel approach may introduce superior results to the drugs known so far by solving major molecular pathology causing Insulin Resistance.

Direct alleviation of the repression of the GLUT4 gene may be achieved by diminishing or preventing AHNAK binding to the GLUT4 promoter, and indirect alleviation may be achieved for example by reducing expression of the AHNAK gene, by interference with one of the signal transduction pathways regulating AHNAK activity such as, but not limited to, e.g., the RAF-MEK-ERK signaling pathway, or by preventing AHNAK from entering the nucleus by modulating, e.g., the PKB/Akt kinase.

In one embodiment, the agent is a small organic molecule or a peptide which may be derived from the AHNAK DNA-binding domain or an analog of said peptide. In one embodiment the DNA-binding domain of the AHNAK protein binds to a sequence encompassing the −212 to −197 bp region of the GLUT4 promoter.

In another embodiment, the agent is an oligonucleotide against an AHNAK gene sequence, said oligonucleotide being selected from the group consisting of: (i) an antisense oligonucleotide, (ii) a microRNA, (iii) an siRNA, and (iv) an oligonucleotide of (i), (ii) or (iii) in the form of a plasmid expressing intracellularly in human cells.

The agent is preferably an siRNA against an AHNAK gene sequence comprising between 15 and 30 nucleotides, preferably between 21 and 23 nucleotides, and most preferably 21 nucleotides.

In a more preferred embodiment, the siRNA is double-stranded and comprises at least one pair of the sequences selected from the group consisting of:

| | |
|---|---|
| (i) Sense strand: | |
| GGAGGUACCUGUUCCUAAAUU, | (SEQ ID NO: 1) |
| Anti sense strand: | |
| 5′-P UUUAGGAACAGGUACCUCCUU; | (SEQ ID NO: 2) |
| (ii) Sense strand: | |
| GGAUAUUUCUCUACCUAAAUU, | (SEQ ID NO: 3) |
| Anti sense strand: | |
| 5′-P UUUAGGUAGAGAAAUAUCCUU; | (SEQ ID NO: 4) |
| (iii) Sense strand: | |
| GACCAAACAUAAAGGGUGAUU, | (SEQ ID NO: 5) |
| Anti sense strand: | |
| 5′-P UCACCCUUUAUGUUUGGUCUU; | (SEQ ID NO: 6) |
| and | |
| (iv) Sense strand: | |
| GGGUUGAGCACAUCAGAUAUU, | (SEQ ID NO: 7) |
| Anti sense strand: | |
| 5′-P UAUCUGAUGUGCUCAACCCUU. | (SEQ ID NO: 8) |

In a most preferred embodiment, the agent consists of a mixture of the pairs of sequences (i)-(iv), namely, a mixture of the siRNAs of the sequences set forth in SEQ ID NO:1 to SEQ ID NO:8.

Antisense agents that may be useful in this invention include oligonucleotides containing modified backbones or non-natural internucleoside linkages. Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′-linked analogs of these, and those having inverted polarity wherein one or more internucleotide linkages is a 3′ to 3′, 5′ to 5′ or 2′ to 2′ linkage.

Oligonucleotides having inverted polarity comprise a single 3′ to 3′ linkage at the 3′-most internucleotide linkage i.e. a single inverted nucleoside residue which may be a base (the nucleobase is missing or has a hydroxyl group in place thereof). Various salts, mixed salts and free acid forms are also included.

Other modified oligonucleotides have backbones that are formed by short chain alkyl or cycloalkyl internucleoside linkages, mixed heteroatom and alkyl or cycloalkyl internucleoside linkages, or one or more short chain heteroatom or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

The preparation of the above modified oligonucleotide backbones is well known to the average person skilled in the art.

The agent of the invention may be administered to a person in need, for example, in a pharmaceutical composition comprising a pharmaceutically acceptable carrier, such as saline or phosphate-buffered saline, and may be administered subcutaneously, intradermally, intratracheally, intranasally, or intravenously.

In another aspect, the present invention relates to a method for detecting a modulator of AHNAK-mediated repression of GLUT4 gene expression, comprising contacting a candidate agent with a mammalian cell transfected with an expression vector containing a heterologous gene, such as a reporter gene, operably linked to a GLUT4 promoter and an additional expression vector containing a fraction of the AHNAK gene encoding a fraction of the AHNAK protein comprising the DNA-binding domain capable of binding to said GLUT4 promoter and repressing its activity, and comparing the level of expression of said heterologous gene in the presence of the agent and in the absence thereof, whereby a modulator of AHNAK mediating repression of GLUT4 gene expression is identified.

The DNA-binding domain of the AHNAK protein binds preferably to a sequence encompassing the −212 to −197 bp region of the GLUT4 promoter.

The mammalian cell for the transfection may be an insulin-responsive cell, such as an adipocyte, a smooth muscle cell, a skeletal muscle cell and a cardiac muscle cell, or a non-insulin responsive cell such as a brain, liver, gut or pancreas cell or a monocyte such as a macrophage.

In the context of the present invention, which aims at alleviating the repression of GLUT4 gene expression, the modulator identified using the above mentioned method should alleviate AHNAK mediated repression of GLUT4 gene expression.

The invention will now be illustrated by the following non-limiting examples.

### Examples

### Experimental Procedures

Reagents. Insulin, LY and arachidonic acid were purchased from SIGMA, Israel. Cell culture reagents were purchased from Biological Industries (Beth-Haemek, Israel).

Isolation of DNA-binding proteins and mass spectrometry. A 5′-biotinylated target DNA oligonucleotide corresponding to GLUT4 promoter region −222/−197 bp was commercially synthesized (Sigma, Israel). Nuclear extracts were prepared from H9C2 myotubes (ATCC CRL-1446), that have been incubated for 24 hrs. in either the absence or presence of arachidonic acid, and cells were then lysed in nuclear extraction buffer (20 mM HEPES [pH 7], 10 mM KCl, 1 mM MgCl₂, 0.5 mM dithiothreitol, 0.1% Triton X-100, 20% glycerol) supplemented with protease inhibitors (Complete Mini, Roche Diagnostics, Germany). Extracts were homogenized, spinned down at 13,000×g for 10 min at 4 C, and the nuclear extract pellets were dissolved in hypotonic extraction buffer containing 420 mM NaCl. Protein level was assessed by BCA method (Pierce, Belgium). Binding assay and protein isolation were preformed using μMACS Factor Finder kit (Miltenyi Biotec, Germany), according to the instructions of the kit. The elute was sent to mass spectrometry analysis at the Proteomics center, at the Technion Institute, Israel. The mass spectrometry data were clustered and analyzed using the Pep-Miner (Beer, Barnea et al. 2004) searching the NR-NCBI database and Sequest software (J. Eng and J. Yates, University of Washington and Finnigan, San Jose).

Expression vectors and luciferase promoter reporters. Expression vectors, pC-DY, pM-DY, and pN-DY, baring the C-terminus, middle domain, and N-terminus of human AHNAK in c-myc-tagged pCDNA3 (Hashimoto, Amagai et al. 1993; Nie, Ning et al. 2000), were a generous gift of Dr. Hashimoto (Department of Dermatology, Kurume University School of Medicine, Kurume, Fukuoka, Japan). The rat GLUT4 promoter (GLUT4-P) reporter and a derived series of progressively 5′-deleted promoter reporters, have been described by us previously (Armoni, Harel et al. 2005). A control synthetic reporter 3×IRS-LUC, containing three repeats of an insulin response element consensus motif in pGL2 was provided by Dr. Eric Tang (Tang, Nunez et al. 1999).

Cell cultures and transient expression assays. In vitro studies were performed in rat embryonal heart-derived H9C2 cells and in primary cultures of rat adipocytes (prepared by us as described in (Armoni, Harel et al. 1995), according to procedures described by us before (Armoni, Harel et al. 2005).

H9C2 myotubes were transfected in the 8-day, fully differentiated state, by the Ca/PO4 method, with 2 μg of GLUT4 promoter reporter DNA, 0-5 μg of expression vectors and 1 μg of pCMV-β-galactosidase. Five hours later cells were glycerol-shocked, and then incubated in differentiation medium for 24 hrs. The next day, the medium was replaced with serum-free medium supplemented with 1% bovine serum albumin and 0-100 μM of arachidonic acid, and the cells were incubated for additional 24 h at 37° C. until harvested.

PRA were transfected by electroporation with 2 μg of GLUT4 promoter reporter DNA, 0-5 μg of expression vectors and 0.5 μg of pCMV-β-galactosidase. One hour later, an equal volume of incubation medium supplemented with 7% bovine serum albumin was added to the DNA-containing medium, and the cells were incubated for an additional 20-24 h at 37° C. arachidonic acid or insulin were included in the medium as indicated. In each experiment, the total amount of DNA transfected was held constant by adding the empty expression vector (pCDNA3), to account for squelching by the promoter itself. Luciferase activity was assayed at room temperature using a luciferase reporter assay kit (Promega Corp, Wisconsin USA) and a Lumat LB9501 luminometer (Berthold Systems, Inc., Nashua, N.H.). Luciferase activity was normalized to β-galactosidase activity as an internal control. Within each experiment, values were expressed as a percentage of the basal GLUT4 promoter activity, i.e. the activity obtained in cells transfected with promoter reporter alone. Cell viability was assessed by trypan blue exclusion. Each experiment was repeated three to four times, with each sample analyzed in quadruplicates.

Immunofluorescence Studies—Human embryonic kidney (HEK) cells were plated in 60 mm plates at a density of 500,000 cells/plate. Cells were incubated for 24 h at 37° C. The next day, cells were washed with magnesium-containing phosphate-buffered saline and transferred to serum-deprived medium supplemented with 2% BSA either with or without Insulin (100 nM). After 24 h, the cells were fixed in 4% paraformaldehyde and stained for indirect immunofluorescence using an anti-AHNAK primary antibody (SC 81048), followed by a FITC-conjugated goat anti-mouse secondary antibody. The nuclei were stained by DAPI.

Chromatin Immunoprecipitation (ChIP). ChIP assays were performed as previously described (Armoni, Harel et al. 2006). Briefly, H9C2 cells were transfected with myc-tagged pC-DY, pM-DY, or pN-DY parts of human AHNAK in pCDNA3. Forty-eight hours post-transfection, DNA and protein were subjected to two-step cross-linking method (Nowak, Tian et al. 2005), then cells were lysed and repeatedly sonicated, to generate DNA fragments of an average size of 500 bp. An aliquot of the whole cell lysate was removed for purification of total DNA, and immunoprecipitations were conducted using either anti-IgG or anti-Myc antibodies. DNA was extracted from the immunoprecipitates, and PCR (26 cycles) was conducted on total DNA and immunoprecipitated DNA with primers corresponding to promoter region −222/−197 bp of the rat GLUT4 promoter. PCR products were analyzed on 2% agarose gel and visualized by ethidium bromide staining in the presence of DNA molecular mass markers.

RNA extraction and quantitative real-time (QRT)-PCR. Total cellular RNA was prepared using a TriReagent kit (Molecular Research Center, Inc., Cincinnati, Ohio) and further purified using RNeasy® columns (Qiagen GmbH, Hilden, Germany). Fluorescent QRT-PCR (Taqman method) was applied to assess GLUT4 and AHNAK mRNA levels, using specific primers and probes that were custom-designed for the Taqman approach (Primer Design, Southampton, UK). Each reaction contained 5 ng of reverse transcribed RNA in 25 μl. Primers and probes for detection of actin and 18S rRNA (Applied Biosystems) were used as an endogenous control to account for differences in the extraction and reverse transcription of total RNA.

AHNAK siRNA. Double-stranded RNAs (dsRNAs) targeting the rat AHNAK were purchased from Dharmacon (Dharmacon, Inc, California, USA). The siRNA (On TARGETplus SMARTpool L-101672-01-0010, Rat Ahnak, XM_{—}574618) used in the experiments is a mixture consisting of the siRNAs of the sequences set forth in SEQ ID NO:1 to SEQ ID NO:8.

PRA were transfected by electroporation with the dsRNA (40 ng in PBS, pH=7.4) as described above. After 24 hrs, cells were harvested and total RNA and protein were obtained. Scrambled non targeting duplex RNA (NT-siRNA) was transfected as control. AHNAK silencing was assessed by QRT-PCR, and normalized to 18S rRNA. GLUT4 expression at protein level was assessed by Western immunoblotting as detailed by us before (Armoni et al., 2002; Armoni et al., 2005).

Statistical Analysis. The data were analyzed statistically using a two-tailed Student's t test analysis for unpaired samples. p values<0.05 were considered statistically significant. The results are expressed as the means±S.E.

### Example 1

### Isolation of AHNAK Protein Attached to GLUT4 Promoter Sequence —222/−197 bp

In a previous work, we identified the −212/−197 bp region of GLUT4-P as mediator of arachidonic acid repression of GLUT4-P (Armoni et al., 2005). Presently, we used a 5′-biotinylated 25-mer corresponding to the −212/−197 bp sequence of GLUT4-P, conjugated to biotin-avidin affinity column, as a bate to identify potential DNA binding protein(s) from nuclear extracts prepared from arachidonic acid-treated H9C2 cells. Mass spectrometry analysis of the eluates revealed 9 peptides resembling the AHNAK/Desmoyokin nucleoprotein, that were attached to the 25-mer bate (data not shown). As AHNAK was previously shown to participate in arachidonic acid-mediated cellular signaling in cardiac muscle (Sekiya et al, 1999; Sussman et al., 2001), we hypothesized that it can also be the protein mediating arachidonic acid-induced GLUT4-P repression. Furthermore, our data show that the area located within −222/−197, which we previously identified as an arachidonic acid response element, is conserved between H9C2 cardiomyotubes and primary rat adipocytes, and deletion of this region resulted in de-repression and activation of the promoter activity (data not shown). This analysis confirms that the same DNA sequence located within this region mediates FFA repression in both cell types—cardiomyocytes and adipocytes.

### Example 2

### Arachidonic Acid and AHNAK Repress GLUT4 Promoter Activity

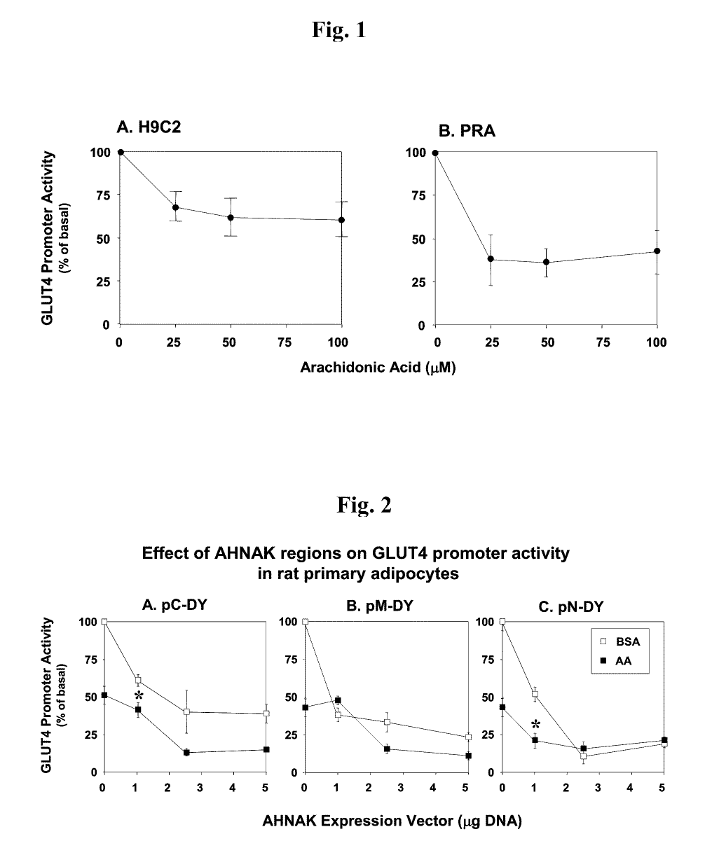

To establish PRA as a suitable cellular model for arachidonic acid studies, parallel experiments were preformed in H9C2 and in PRA. Both cell types were transfected with GLUT4-P reporter, and incubated with ambient arachidonic acid levels as indicated. Our data show that in both cell types, arachidonic acid dose-dependently repressed transcription from GLUT4-P (FIGS. 1A-B), with major effect observed in PRA (FIG. 1B).

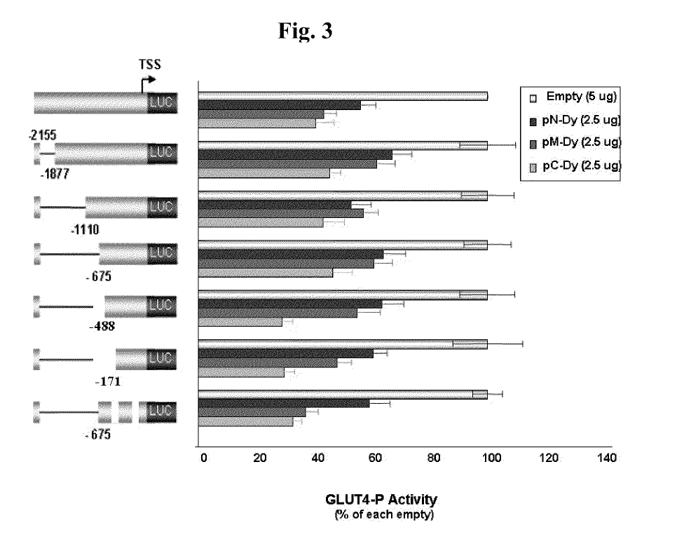

AHNAK (“giant” in Hebrew) is the biggest protein ever cloned, notable for its exceptional size of ˜700 kDa (Shtivelman et al., 1992). To investigate which domain(s) of AHNAK protein are responsible for its binding to, and regulation of GLUT4-P expression, we used three different eukaryotic expression plasmids, pC-DY, pM-DY, and pN-DY, which were previously shown to express C-terminus, middle domain, and N-terminus of this molecule, respectively, upon transfection to cells (Hashimoto et al., 1993; Nie et al., 2000). We found that transient expression of either pC-DY, pM-DY or pN-DY into PRA, dose-dependently repressed basal activity of the co-transfected GLUT4-P, to a maximum of 67%±5 (p≦0.05), 74%±3 (p≦0.05) and 65%±5 (p≦0.05), respectively (FIGS. 2A-C). Furthermore, when combined with arachidonic acid treatment (100 μM, 24 hrs), overexpression of either pC-DY, pM-DY, or pN-DY resulted in enhanced repression of GLUT4-P activity, by as much as 85%±1 (p≦0.05), 89%±3 (p≦0.05) and 79%±2 (p≦0.05), promoter repression, respectively. These data suggest that an additive effect exists between AHNAK and arachidonic acid, acting together to repress GLUT4 expression on PRA.

### Example 3

### Detection of Cis-Elements on GLUT-P That Mediate its Repression by AHNAK

To identify cis-elements on the GLUT4 promoter, that may serve as potential AHNAK binding sites, we performed a progressive 5′-deletion analysis of the full-length GLUT4-P reporter. Cells were transfected with a series of 5′-deleted promoter reporters (FIG. 3A), along with either pC-DY, pM-DY, or pN-DY of AHNAK (FIG. 3B). As a reference for maximal GLUT4-P activity, cells were transfected with GLUT4-promoters but not with an AHNAK expression vector. We found that deleting GLUT4-P area located within −1110/−675 was sufficient to mediate AHNAK C terminal maximal repression of the GLUT4-P. AHNAK middle part maximal repression requires deleting two adjacent areas in GLUT4-P; an area located within −1110/−675 as well as −675/−488. Only when the two areas are deleted, AHNAK middle part was able to induce further repression of GLUT-4 promoter.

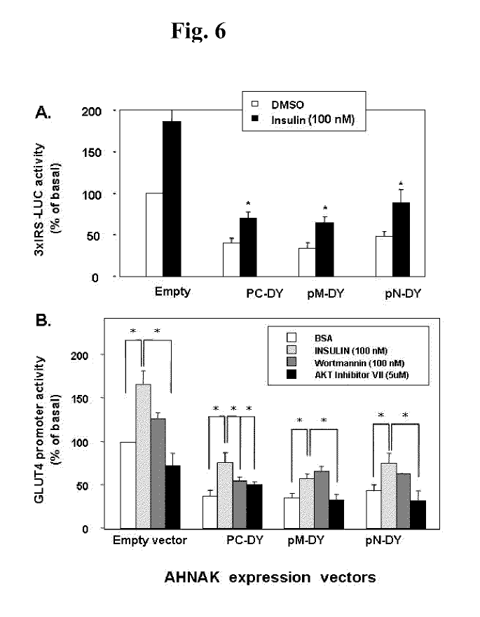

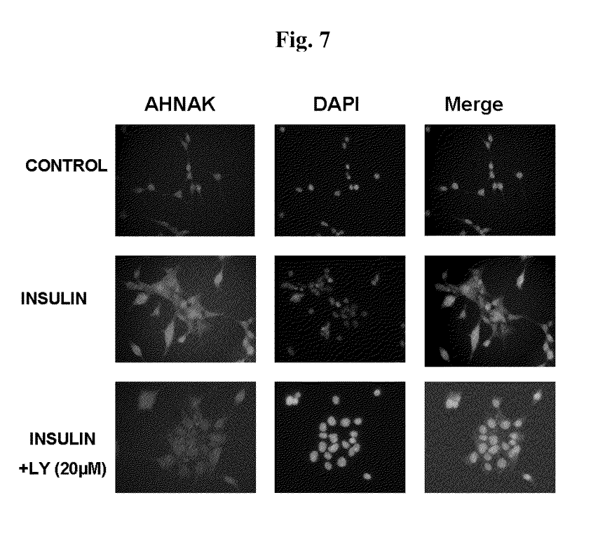

Since our findings in this work confirmed that AHNAK mediates FFA repression on GLUT4-P (FIGS. 2 and 5), and in order to precisely identify response elements mediating AHNAK repression of the GLUT4-P, we further deleted two specific consensus binding sites that were previously shown to mediate FFA repression on GLUT4-P (Armoni et al. 2005). Indeed, deleting areas −612/−587 as well as −222/−197 in GLUT4-P resulted in similar maximal repression of the promoter. Consensus binding motives for PPAR exist in these areas (Armoni et al., 2005). Thus, AHNAK repression of GLUT4-P may be mediated via PPAR.

### Example 4

### AHNAK Middle Part Binds to GLUT4-P Region −222/−197 in Cellulo

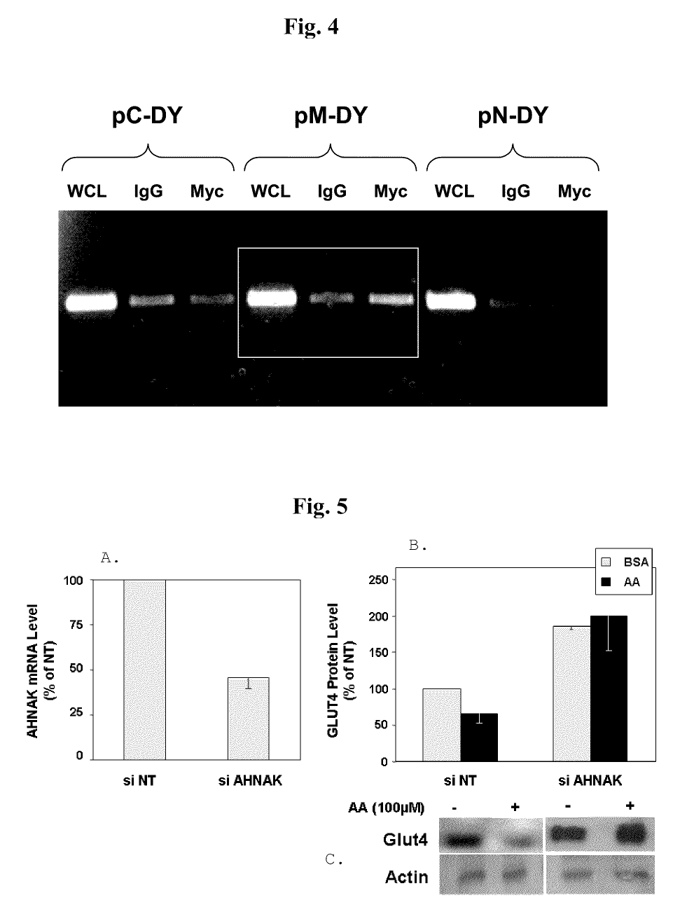

To determine whether AHNAK binds to GLUT4-P region −222/−197 bp in cellulo, we performed ChIP analysis in H9C2 cells that have been transfected with c-myc-tagged pC-DY, pM-DY, or pN-DY vectors. DNA-protein complexes were precipitated with either anti-c-myc or anti-IgG antibodies (control), then subjected to PCR analysis using primers encompassing the −222/−197 bp region. The results show specific binding of AHNAK middle part to −222/−197 GLUT4-P region; no specific binding could be observed for either C-terminal or N-terminal parts (FIG. 4). These results confirm the mass spectrometry data, that AHNAK middle part binds GLUT4-P thus regulating its activity.

### Example 5

### AHNAK siRNA Enhances Expression and Protects from Arachidonic Acid-Induced Repression of the GLUT4 Gene

Since our in vitro results indicated that AHNAK over expression decreased GLUT4-P activity, we examined the effects of AHNAK gene silencing on the endogenous expression of GLUT4. PRA were transfected with siRNA for AHNAK and non targeting scrambled RNA (NT). Twenty four hrs after transfection, RNA and total protein were extracted, and endogenous expression of AHNAK and GLUT4 were assessed. Cells transfected with non targeting siRNA were used as control. We found that levels of AHNAK mRNA were reduced to approximately 60%±3 (p≦0.05) after 24 hrs, resulting in ˜2-fold GLUT4 protein levels (FIG. 5A). To our best knowledge, this is the first indication that AHNAK regulates endogenous GLUT4 protein expression.

In the presence of arachidonic acid, AHNAK activates PLCγ1 (Sekiya et al., 1999). PLCγ also participates in insulin signaling and glucose transport (Lorenzo et al., 2002). In order to ascertain that AHNAK participates in arachidonic acid induced GLUT4 gene repression, we examined whether AHNAK silencing could prevent the arachidonic acid-induced decrease in GLUT4 protein levels. PRA were transfected with either NT or AHNAK siRNA, in the absence or presence of arachidonic acid (100 μM, 24 hrs), and GLUT4 protein levels were then assessed using Western blot. We found that while in NT-transfected cells arachidonic acid incubation resulted in ˜40%±3 (P≦0.05) decrease in GLUT4 protein levels, AHNAK silencing abrogated this effect (FIG. 5B). Thus, our data indicate that AHNAK silencing not only enhances GLUT4 gene expression, but also protects it from arachidonic acid-induced repression. Taken together, this strongly suggests that AHNAK is indeed the mediator of arachidonic acid detrimental effects on GLUT4 expression. Noteworthy is the fact that in arachidonic acid-untreated cells AHNAK silencing enhanced cellular GLUT4 protein to about 185%±13 (P≦0.05).

Suggested Mechanism for AHNAK and arachidonic acid regulation of GLUT4 Expression. AHNAK was previously shown to be phosphorylated by PKB/Akt, which results in its nuclear exclusion (Sussman et al., 2001). PI3K-Akt signaling is activated by insulin and plays a pivotal role in GLUT4 translocation (Saltiel and Kahn 2001). Beyond the short term effects, insulin signaling also regulates gene transcription via specific IRE sequences (Insulin Response Elements) located in promoters of genes (Accili and Arden, 2004). We have shown that FOXO1 (previously FKHR), a transcription factor from the forkhead family, regulates GLUT4 gene transcription in an insulin-dependent and tissue-specific manner (Armoni et al., 2002). FOXO1 was previously shown to bind in vitro to IREs in the insulin-like growth factor-binding protein 1 promoter (Accili and Arden, 2004) and to activate transcription from a reporter plasmid containing multiple copies of the IRE (Tang, Nunez et al. 1999). Screening the −222/−197 bp region of GLUT4-P for consensus motifs, we found that it indeed contains an IRE response element (data not shown). Thus, we examined whether AHNAK, like FOXO1, represses GLUT4-P via IRE elements, and found that all three parts of AHNAK repressed transcription from the IRE of the 3×IRS-LUC reporter by more that 50%. Interestingly, insulin administration curtailed this repression. AKT pharmacologic inhibitors (which inhibit down stream signaling of insulin pathway), abrogated insulin effect and resulted in re-repression of the promoter.

Insulin administration results in AHNAK nuclear exclusion in HEK cells. In order to examine whether insulin affects AHNAK cellular distribution, HEK cells were chosen mostly due to easy staining procedure, and were incubated for 24 hrs in a serum free medium containing insulin (100 nM) or in insulin free medium for control. After 24 hrs endogenous AHNAK was stained by immunoflorescence (FIG. 6). Our results show that while in control cells, AHNAK is mostly located in the nucleus, while in insulin stimulated cells, AHNAK subcellular distribution alters, and most of it translocates to the cytosel. Inhibition of PI3K by LY, i.e disrupting, insulin signaling cascade, resulted in AHNAK nuclear re-distribution, in resemblance to control cells

To best of our knowledge, this is the first indication for insulin role in AHNAK subcellular distribution.

### Example 7

### Patients and Tissue Biopsies for in Vivo Studies

The tissue-specific expression of GLUT4 and AHNAK is studied in vivo in various adipose tissues under physiological conditions as well as in insulin resistant states. Adipose tissue biopsies are obtained from patients with various degrees of obesity and/or hyperglycaemia and/or diabetes type 2 (DM2), hypertensive or patients with metabolic syndrome and matched euglycemic controls with no previous thyroid, renal, adrenal, or liver abnormalities. After an overnight fast, ˜10 g of abdominal sc. and/or omental adipose tissues are obtained from these patients while undergoing elective surgery. Isolated adipose cells are prepared from the tissue samples according to procedures well established by us as published before (Armoni et al., 1987; Karnieli et al., 1989). To extend our investigation to other insulin-responsive tissues, cardiac muscle and skeletal muscle tissues are also obtained from patients undergoing elective coronary arteries bypass grafts or orthopedic operations, respectively. Blood samples are obtained from the patients for the determination of glucose, insulin, C-peptide levels, HbA1c, and lipids level using standard lab procedures. Further, macrophages are isolated from the same patients. Muscle biopsies are also obtained from normal volunteers, obese and diabetic patients before and after endurance exercise.

### Example 8

### Monocyte Isolation

AHNAK and GLUT4 were assessed in monocytes isolated according to the method published previously (Boyum, 1968). Twenty ml of blood were taken from a peripheral vein and placed in a heparinized tube on ice. The blood was layered on 10 ml of histophaque 1077 (Sigma) gradient and centrifuged for 30 minute. The middle layer (“buffy coat”) containing the leucocytes was collected and washed twice with PBS. The cells were resuspended in DMEM 2% FCS with 100 units/ml penicillin and 0.1 mg/ml streptomycin, and plated in a 10 mm tissue culture dish. After one hour of incubation at 37° C., the monocytes adhered to the plates. The medium was then aspirated and the monocytes harvested. A sample was taken for mRNA quantitation using qRT-PCR and other sample was lyzed for immunoblotting studies. We obtained preliminary data in mouse-derived macrophages (J774) proving the existence of AHANK transcripts in these cells (data not shown).

### Discussion

AHNAK was identified as a giant protein that participates in arachidonic acid metabolism, calcium signaling at the plasma membrane (Haase et al., 1999), skeletal muscle regeneration (Huang et al., 2007), and cytoskeletal functions (Hohaus et al., 2002; Haase et al., 2004) Importantly, a 6-fold up-regulation of AHNAK mRNA was seen in white adipose tissue of ZDF rats at the very start of obesity, after the first week of high fat feeding (Li et al., 2002). However its role in insulin resistance was never studied. According to the present invention, we demonstrate that the AHNAK protein participates in the regulation of glucose homeostasis by suppressing the GLUT4 gene expression, via direct binding to GLUT4-P region −222/−197 bp and probably indirectly to other regions of the GLUT4 promoter, by association with other transcription factors. We also suggest that AHNAK is involved in arachidonic acid-mediated signaling that leads to decrease of cellular GLUT4 gene expression (Armoni et al., 2005), thus introducing a novel role for AHNAK in the pathogenesis of insulin resistance associated with obesity, lipotoxicity, hypertension, type 2 diabetes and metabolic syndrome.

How the AHNAK conveys its effect on the glucose transport system has not been previously studied. AHNAK nuclear transport/exclusion is governed by PKB/Akt phosphorylation on a nuclear export signal located at the C terminus of AHNAK (Sussman et al., 2001). According to our data, a shift of AHNAK nuclear distribution and cytosol translocation occurs in HEK cells incubated with insulin (FIG. 6). Once in the nucleus, AHNAK may interfere with insulin action by repressing GLUT4 transcription via binding to IRE elements present in the GLUT4-P. Our data show that it is the middle part of AHNAK that binds to cis-element(s) located in −222/−197 bp region of GLUT4-P, which contains an IRE element (FIG. 4). As AHNAK nuclear localization is PKB/Akt dependent, impairment in insulin signaling cascade, known to occur in cases of obesity, and high levels of blood fatty acids and/or proinflammatory cytokines, at steps upward of PKB/Akt (Zick 2005), results in AHNAK nuclear sequestration leading to repression of GLUT4 gene expression. Further, as AHNAK, in association with calcium channels or actin-based cytoskeleton, has been observed in various tissues (Haase et al., 1999; Hohaus et al., 2002; Gentil et al., 2003; Haase et al., 2004), a role for this protein is emerging in altering signaling pathways. AHNAK participates in arachidonic acid signaling by promoting a physical interaction between AHNAK and PLCγ1, and the activation by AHNAK and arachidonic acid was mainly attributable to reduction in the enzyme's apparent Km toward its substrate phosphatidylinositol 4,5-bisphosphate (Sekiya et al., 1999). Our data suggest a that AHNAK is a down stream mediator for FFA signaling pathways that lead to down-regulation of GLUT4 gene expression (FIGS. 2 and 5)

To further investigate whether AHNAK and arachidonic acid exert their detrimental effects via the same mechanism, we examined the effects of AHNAK gene silencing on GLUT4 expression. AHNAK silencing by 60% not only lead to 2-fold increased GLUT4 protein levels, but also protected the cells from arachidonic acid-induced GLUT4 repression (FIGS. 5A-B). Thus, AHNAK is suggested as a new mediator of the detrimental effects of high FFA levels and hyperlipidemia associated with lipotoxicity and insulin resistance.

Akt activation and subsequence phosphorylation of downstream substrates on serine residues is a well-established step in insulin signaling to GLUT4 translocation and function (see review (Saltiel and Kahn 2001)). As the AHNAK is phosphorylated by PKB/Akt (Sussman, Stokoe et al. 2001), we examined the cross-talk between insulin signaling and AHNAK regulation of GLUT4-P. We observed that insulin administration partially rescued the activity of promoters containing IRE, such as the artificial 3×IRE-LUC or GLUT4-P, from repression caused by AHNAK fragments (FIG. 6). These results point out that AHNAK may interact with the insulin pathway in addition to mediating arachidonic acid signaling.

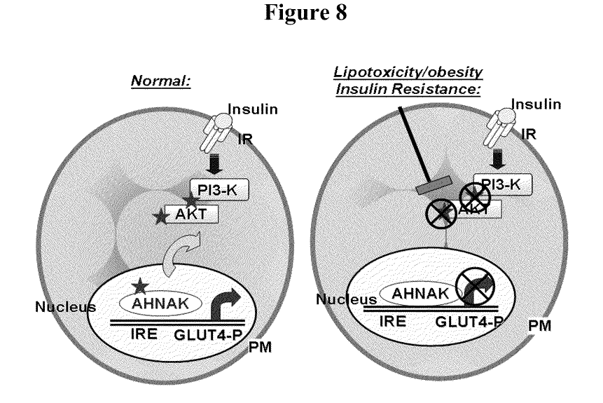

Based on the data presented here and those gathered from the literature, we suggest the following model for the role of AHNAK in the regulation of glucose metabolism (FIG. 8). In the basal state, AHNAK recycles between the nucleus and the cytoplasm. In the nucleus, the AHNAK protein can bind to IRE sequences located with in GLUT4 promoter resulting in GLUT4 gene repression. Upon insulin stimulation PKB/Akt, phosphorylates AHNAK on serine 5535 residue as described previously (Sussman et al., 2001), leading to AHNAK nuclear as presented in our data, exclusion and alleviation of GLUT4 promoter suppression. Hyperlipidemia (lipotoxicity state) impedes insulin signaling by activation of protein kinase C_{θ} and reduction of IRS-1 phosphorylation (Yuan et al., 2001), resulting in decreased PI3K-PKB/Akt phosphorylation cascade activity and diminished AHNAK phosphorylation. In its dephosphorylated state the AHNAK resides within the nucleus, repressing GLUT4 gene expression.

Several facts suggest that AHNAK mRNAs levels are increased in insulin resistance states in vivo: the observation that AHNAK mRNA is 6-fold increased in adipocytes at the early phases of obesity induction in Zucker rats (Li, Yu et al. 2002); according to our preliminary data AHNAK mRNA is increased in muscle tissue from diabetic rats and reduced upon insulin therapy (not shown). Moreover, high expression of AHNAK was associated with a low VO2 max and poor muscle fitness in aged skeletal muscle biopsies, as well as a decrease with exercise and an increase with aging. Preliminary studies made in our lab in human primary macrophages indicate a relation between AHNAK levels, hypertension and metabolic syndrome (not shown).

Thus, increased cellular level of AHNAK and aberrant nuclear localization would lead to reduction in cellular GLUT4. Our data support the hypothesis that AHNAK is a novel regulator of GLUT4 gene expression that participates in the pathophysiology of insulin resistance. Since silencing of AHNAK expression leads to ˜2 fold increase in cellular GLUT4 level, AHNAK is introduced as a novel molecular target for therapy in insulin resistance states like obesity and diabetes.

### REFERENCES

Accili, D. and K. C. Arden (2004). “FoxOs at the crossroads of cellular metabolism, differentiation, and transformation.” Cell 117(4): 421-6.

Alkhateeb, H., A. Chabowski, et al. (2007). “Two phases of palmitate-induced insulin resistance in skeletal muscle: impaired GLUT4 translocation is followed by a reduced GLUT4 intrinsic activity.” Am J Physiol Endocrinol Metab 293(3): E783-793.

Armoni, M., C. Harel, et al. (2005). “Free Fatty Acids Repress the GLUT4 Gene Expression in Cardiac Muscle via Novel Response Elements.” J. Biol. Chem. 280(41): 34786-34795.

Armoni, M., C. Harel, et al. (2007). “Transcriptional regulation of the GLUT4 gene: from PPAR-gamma and FOXO1 to FFA and inflammation.” Trends Endocrinol Metab 18(3): 100-7.

Armoni, M., M. J. Quon, et al. (2002). “PAX3/Forkhead Homolog in Rhabdomyosarcoma Oncoprotein Activates Glucose Transporter 4 Gene Expression in Vivo and in Vitro.” J Clin Endocrinol Metab 87(11): 5312-24.

Armoni, M., R. Rafaeloff, et al. (1987). “Sex differences in insulin action on glucose transport and transporters in human omental adipocytes.” J Clin Endocrinol Metab 65(6): 1141-6.

Boden, G. (1997). “Role of fatty acids in the pathogenesis of insulin resistance and NIDDM.” Diabetes 46(1): 3-10.

Boyum, A. (1968). “Separation of leukocytes from blood and bone marrow. Introduction.” Scand J Clin Lab Invest Suppl 97: 7.

Charron, M. J., E. B. Katz, et al. (1999). “GLUT4 gene regulation and manipulation.” J Biol Chem 274(6): 3253-6.

Gentil, B. J., C. Delphin, et al. (2003). “Expression of the giant protein AHNAK (desmoyokin) in muscle and lining epithelial cells.” J Histochem Cytochem 51(3): 339-48.

Graham, T. E., Q. Yang, et al. (2006). “Retinol-binding protein 4 and insulin resistance in lean, obese, and diabetic subjects.” N Engl J Med 354(24): 2552-63.

Griffin, M. E., M. J. Marcucci, et al. (1999). “Free fatty acid-induced insulin resistance is associated with activation of protein kinase C theta and alterations in the insulin signaling cascade.” Diabetes 48(6): 1270-4.

Haase, H., I. Pagel, et al. (2004). “The carboxyl-terminal ahnak domain induces actin bundling and stabilizes muscle contraction.” Faseb J 18(7): 839-41.

Haase, H., T. Podzuweit, et al. (1999). “Signaling from beta-adrenoceptor to L-type calcium channel: identification of a novel cardiac protein kinase A target possessing similarities to AHNAK.” Faseb J 13(15): 2161-72.

Hashimoto, T., M. Amagai, et al. (1993). “Desmoyokin, a 680 kDa keratinocyte plasma membrane-associated protein, is homologous to the protein encoded by human gene AHNAK.” J Cell Sci 105 (Pt 2): 275-86.

Hohaus, A., V. Person, et al. (2002). “The carboxyl-terminal region of ahnak provides a link between cardiac L-type Ca2+ channels and the actin-based cytoskeleton.” Faseb J 16(10): 1205-16.

Huang, Y., S. H. Laval, et al. (2007). “AHNAK, a novel component of the dysferlin protein complex, redistributes to the cytoplasm with dysferlin during skeletal muscle regeneration.” Faseb J 21(3): 732-42.

Jove, M., A. Planavila, et al. (2006). “Palmitate induces tumor necrosis factor-alpha expression in C2Cl2 skeletal muscle cells by a mechanism involving protein kinase C and nuclear factor-kappaB activation.” Endocrinology 147(1): 552-61.

Karnieli, E., R. Moscona, et al. (1989). “Discrepancy between glucose transport and transporters in human femoral adipocytes.” Am J Physiol 256(1 Pt 1): E179-85.

Lee, I. H., H. J. Lim, et al. (2008). “Ahnak protein activates PKC through dissociation of PKC-PP2A complex.” J Biol Chem.

Lee, I. H., J. O. You, et al. (2004). “AHNAK-mediated activation of phospholipase C-gamma1 through protein kinase C.” J Biol Chem 279(25): 26645-53.

Li, J., X. Yu, et al. (2002). “Gene expression profile of rat adipose tissue at the onset of high-fat-diet obesity.” Am J Physiol Endocrinol Metab 282(6): E1334-41.

Li, X. L., S. Aou, et al. (2002) “Impairment of long-term potentiation and spatial memory in leptin receptor-deficient rodents.” Neuroscience 113(3): 607.

Lorenzo, M., T. Teruel, et al. (2002). “PLCgamma participates in insulin stimulation of glucose uptake through activation of PKCzeta in brown adipocytes.” Exp Cell Res 278(2): 146-57.

Nie, Z., W. Ning, et al. (2000). “C-Terminus of desmoyokin/AHNAK protein is responsible for its translocation between the nucleus and cytoplasm.” J Invest Dermatol 114(5): 1044-9.

Petersen, K. F. and G. I. Shulman (2006). “Etiology of insulin resistance.” Am J Med 119(5 Suppl 1): S10-6.

Saltiel, A. R. and C. R. Kahn (2001). “Insulin signaling and the regulation of glucose and lipid metabolism.” Nature 414(6865): 799.

Sekiya, F., Y. S. Bae, et al. (1999). “AHNAK, a protein that binds and activates phospholipase C-gamma1 in the presence of arachidonic acid.” J Biol Chem 274(20): 13900-7.

Sessler, A. M. and J. M. Ntambi (1998). “Polyunsaturated fatty acid regulation of gene expression.” J Nutr 128(6): 923-6.

Shepherd, P. R. and B. B. Kahn (1999). “Glucose transporters and insulin action—implications for insulin resistance and diabetes mellitus.” N Engl J Med 341(4): 248-57.

Shtivelman, E., F. E. Cohen, et al. (1992). “A human gene (AHNAK) encoding an unusually large protein with a 1.2-microns polyionic rod structure.” Proc Natl Acad Sci USA 89(12): 5472-6.

Stiff, T., E. Shtivelman, et al. (2004). “AHNAK interacts with the DNA ligase IV-XRCC4 complex and stimulates DNA ligase IV-mediated double-stranded ligation.” DNA Repair (Amst) 3(3): 245-56.

Sussman, J., D. Stokoe, et al. (2001). “Protein kinase B phosphorylates AHNAK and regulates its subcellular localization.” J Cell Biol 154(5): 1019-30.

Tataranni, P. A. and E. Ortega (2005). “A Burning Question: Does an Adipokine-Induced Activation of the Immune System Mediate the Effect of Overnutrition on Type 2 Diabetes?” Diabetes 54(4): 917-927.

Wang, X. L., L. Zhang, et al. (2006). “Free Fatty Acids Inhibit Insulin Signaling-Stimulated Endothelial Nitric Oxide Synthase Activation Through Upregulating PTEN or Inhibiting Akt Kinase.” Diabetes 55(8): 2301-2310.

Yang, T. T., H. Y. Suk, et al. (2006). “Role of transcription factor NFAT in glucose and insulin homeostasis.” Mol Cell Biol 26(20): 7372-87.

Yu, C., Y. Chen, et al. (2002). “Mechanism by which fatty acids inhibit insulin activation of insulin receptor substrate-1 (IRS-1)-associated phosphatidylinositol 3-kinase activity in muscle.” J Biol Chem 277(52): 50230-6.

Yuan, M., N. Konstantopoulos, et al. (2001). “Reversal of obesity- and diet-induced insulin resistance with salicylates or targeted disruption of Ikkbeta.” Science 293(5535): 1673-7.

1. An agent capable of alleviating AHNAK-mediated repression of GLUT4 gene expression, wherein said agent is:
(a) a small organic molecule;
(b) a peptide, or
(c) an oligonucleotide against an AHNAK gene sequence, wherein said oligonucleotide is selected from the group consisting of:
(i) an antisense oligonucleotide;
(ii) a microRNA;
(iii) an siRNA; and
(iv) an oligonucleotide of (i), (ii) or (iii) in the form of a plasmid expressing intracellularly in human cells.

2. The agent according to claim 1, wherein said peptide of (b) is derived from the AHNAK DNA-binding domain or an analog of said peptide.

3. The agent according to claim 2, wherein the DNA-binding domain of the AHNAK protein binds to a sequence encompassing the −212 to −197 bp region of the GLUT4 promoter.

4. The agent according to claim 2, wherein said agent is an siRNA against an AHNAK gene sequence comprising between 15 and 30 nucleotides.

5. The agent according to claim 4, wherein said siRNA comprises between 21 and 23 nucleotides.

6. The agent according to claim 5, wherein said siRNA consists of 21 nucleotides.

7. The agent according to claim 6, wherein said siRNA comprises at least one pair of sequences selected from the group consisting of:

| | |
|---|---|
| (i) Sense strand: | |
| GGAGGUACCUGUUCCUAAAUU | (SEQ ID NO: 1) |
| Anti sense strand: | |
| 5′-P UUUAGGAACAGGUACCUCCUU; | (SEQ ID NO: 2) |
| (ii) Sense strand: | |
| GGAUAUUUCUCUACCUAAAUU | (SEQ ID NO: 3) |
| Anti sense strand: | |
| 5′-P UUUAGGUAGAGAAAUAUCCUU; | (SEQ ID NO: 4) |
| (iii) Sense strand: | |
| GACCAAACAUAAAGGGUGAUU | (SEQ ID NO: 5) |
| Anti sense strand: | |
| 5′-P UCACCCUUUAUGUUUGGUCUU; | (SEQ ID NO: 6) |
| and | |
| (iv) Sense strand: | |
| GGGUUGAGCACAUCAGAUAUU | (SEQ ID NO: 7) |
| Anti sense strand: | |
| 5′-P UAUCUGAUGUGCUCAACCCUU. | (SEQ ID NO: 8) |

8. The agent according to claim 7, wherein said siRNA consists of the mixture of the pairs of sequences of (i) to (v) of SEQ ID NO:1 to SEQ ID NO:8.

9. A method for prevention, treatment, alleviation or a combination thereof of insulin resistance associated with obesity, lipotoxicity, hypertension, metabolic syndrome and type 2 diabetes, comprising administering to an individual in need a therapeutically effective amount of an agent capable of alleviating AHNAK phosphoprotein-mediated repression of GLUT4 gene expression, wherein said agent is:
(a) a small organic molecule;
(b) a peptide, or
(c) an oligonucleotide against an AHNAK gene sequence, wherein said oligonucleotide is selected from the group consisting of:
(i) an antisense oligonucleotide;
(ii) a microRNA;
(iii) an siRNA; and
(iv) an oligonucleotide of (i), (ii) or (iii) in the form of a plasmid expressing intracellularly in human cells.

10. The method according to claim 9, wherein said peptide of (b) is derived from the AHNAK DNA-binding domain or an analog of said peptide.

11. The method according to claim 10, wherein the DNA-binding domain of the AHNAK protein binds to a sequence encompassing the −212 to −197 bp region of the GLUT4 promoter.

12. The method according to claim 9, wherein said agent is an siRNA against an AHNAK gene sequence comprising between 15 and 30 nucleotides.

13. The method according to claim 12, wherein said siRNA comprises between 21 and 23 nucleotides.

14. The method according to claim 13, wherein said siRNA consists of 21 nucleotides.

15. The method according to claim 14 wherein said siRNA comprises at least one pair of sequences selected from the group consisting of:

| | |
|---|---|
| (i) Sense strand: | |
| GGAGGUACCUGUUCCUAAAUU | (SEQ ID NO: 1) |
| Anti sense strand: | |
| 5′-P UUUAGGAACAGGUACCUCCUU; | (SEQ ID NO: 2) |
| (ii) Sense strand: | |
| GGAUAUUUCUCUACCUAAAUU | (SEQ ID NO: 3) |
| Anti sense strand: | |
| 5′-P UUUAGGUAGAGAAAUAUCCUU; | (SEQ ID NO: 4) |
| (iii) Sense strand: | |
| GACCAAACAUAAAGGGUGAUU | (SEQ ID NO: 5) |
| Anti sense strand: | |
| 5′-P UCACCCUUUAUGUUUGGUCUU; | (SEQ ID NO: 6) |
| and | |
| (iv) Sense strand: | |
| GGGUUGAGCACAUCAGAUAUU | (SEQ ID NO: 7) |
| Anti sense strand: | |
| 5′-P UAUCUGAUGUGCUCAACCCUU. | (SEQ ID NO: 8) |

16. The method according to claim 15, wherein said siRNA consists of a mixture of the pairs of sequences of (i) to (v) of the SEQ ID NO:1 to SEQ ID NO:8.

17. A method for detecting a modulator of AHNAK-mediated repression of GLUT4 gene expression, comprising contacting a candidate agent with a mammalian cell transfected with an expression vector containing a heterologous gene, such as a reporter gene, operably linked to a GLUT4 promoter and an additional expression vector containing a fraction of the AHNAK gene encoding a fraction of the AHNAK protein comprising the DNA-binding domain capable of binding to said GLUT4 promoter and repressing its activity, and comparing the level of expression of said heterologous gene in the presence of the candidate agent and in the absence thereof, whereby a modulator of AHNAK-mediated repression of GLUT4 gene expression is identified.

18. The method according to claim 17, wherein said heterologous gene is a reporter gene.

19. The method according to claim 17, wherein the DNA-binding domain of the AHNAK protein binds to a sequence encompassing the −212 to −197 bp region of the GLUT4 promoter.

20. The method according to claim 17, wherein said mammalian cell is an insulin-responsive cell selected from the group consisting of an adipocyte, a smooth muscle cell, a skeletal muscle cell and a cardiac muscle cell, or a non-insulin responsive cell selected from the group consisting of brain, liver, gut or pancreas cell.

21. The method according to claim 17, wherein said modulator alleviates AHNAK phosphoprotein-mediated repression of GLUT4 gene expression.


SEQUENCE LISTING (8 sequences)

SEQ ID NO: 1 - RNA, 21 nt, Artificial Sequence
  Synthetic
ggagguaccuguuccuaaauu

SEQ ID NO: 2 - RNA, 21 nt, Artificial Sequence
  Synthetic
uuuaggaacagguaccuccuu

SEQ ID NO: 3 - RNA, 21 nt, Artificial Sequence
  Synthetic
ggauauuucucuaccuaaauu

SEQ ID NO: 4 - RNA, 21 nt, Artificial Sequence
  Synthetic
uuuagguagagaaauauccuu

SEQ ID NO: 5 - RNA, 21 nt, Artificial Sequence
  Synthetic
gaccaaacauaaagggugauu

SEQ ID NO: 6 - RNA, 21 nt, Artificial Sequence
  Synthetic
ucacccuuuauguuuggucuu

SEQ ID NO: 7 - RNA, 21 nt, Artificial Sequence
  Synthetic
ggguugagcacaucagauauu

SEQ ID NO: 8 - RNA, 21 nt, Artificial Sequence
  Synthetic
uaucugaugugcucaacccuu